(19) United States
(12) Patent Application Publication
(10) Pub. No.: US 2026/0248502 A1
(43) Pub. Date: Aug. 27, 2026

(54) Powered Surgical Device With Switch to Control Automatic Retraction

(71) Applicant: Covidien LP, Mansfield, MA (US)

(72) Inventor: Chirong LIU, Shanghai (CN)

(73) Assignee: Covidien LP, Mansfield, MA (US)

(21) Appl. No.: 18/877,153

(22) PCT Filed: Jul. 1, 2022
(86) PCT No.: PCT/CN2022/103271

§ 371 Date: Dec. 19, 2024

(51) Int. Cl. A61B 17/068; A61B 17/00

(52) U.S. Cl. CPC A61B 17/068; A61B 2017/00017; A61B 2017/00398

(57) ABSTRACT

A powered surgical device includes a handle assembly (12, 312, 512) having a rack (56, 356, 556), a first switch (62) for initiating clamping and firing of the surgical device, and a second switch (68, 368, 568) for initiating automatic retraction of the rack (56, 356, 556). The second switch (68, 368, 568) is controlled by a switch control mechanism (80, 380, 580) that is engaged with the rack (56, 356, 556) in the handle assembly (12, 312, 512) to initiate automatic retraction of the rack (56, 356, 556) when firing is completed.

### FIELD

This disclosure is directed to powered surgical devices and, more particularly, to powered surgical stapling devices with automatic retraction after firing.

### BACKGROUND

Various types of surgical devices used to endoscopically treat tissue are known in the art, and are commonly used, for example, for closure of tissue or organs in transection, resection, and anastomoses procedures, for occlusion of organs in thoracic and abdominal procedures, and for electrosurgically fusing or sealing tissue.

One example of such a surgical device is a surgical stapling device. Typically, surgical stapling devices include a tool assembly having an anvil assembly and a cartridge assembly, and a drive assembly. The drive assembly includes a flexible drive beam and a clamp member that is supported on a distal end of the drive beam. The drive assembly is movable to advance the clamp member through the tool assembly to move the tool assembly from an open position to a clamped position, and to advance an actuation sled and knife through the cartridge assembly to eject staples from the cartridge assembly and to cut tissue clamped between the anvil and cartridge assemblies.

Surgical stapling devices can be manually actuated devices or powered stapling devices. Powered stapling devices include one or more actuation buttons to activate a motor to initially advance the drive assembly and move the tool assembly from the open position to a clamped position, and subsequently advance the actuation sled and knife through the tool assembly to fire the stapling device, i.e., eject staples from the cartridge assembly and cut tissue clamped between the cartridge and anvil assemblies. Such devices include a first switch that is manually actuated to advance the drive assembly to move the tool assembly to the clamped position and to fire the stapling device, and a second switch that is manually actuated to unclamp the tool assembly and to retract the drive assembly after firing. Some stapling devices include a third switch that stops advancement of the drive assembly after clamping is completed, and a fourth switch that stops retraction of the drive assembly when the drive assembly is fully retracted after firing is completed.

A continuing need exists in the art for a powered stapling device that will automatically retract the drive assembly after the stapling device is fired.

### SUMMARY

This application is directed to a powered surgical device includes a handle assembly having a first switch for initiating clamping and firing of the surgical device and a second switch for initiating automatic retraction. The second switch is controlled by a switch control mechanism that is engaged with a rack in the handle assembly to initiate automatic retraction when firing is completed.

Aspects of the disclosure are directed to a powered handle assembly including an outer housing defining a cavity. an inner housing secured within the cavity, a rack, a drive assembly, a first switch, an automatic retraction switch, and an automatic retraction switch control mechanism. The inner housing defines a longitudinally extending path through the inner housing. The rack is positioned along the longitudinally extending path of the inner housing and includes a first engagement surface. The rack is movable between retracted and advanced positions. The drive assembly is supported by the inner housing and is engaged with the rack to move the rack between the retracted and advanced positions. The first switch is supported within the cavity and is coupled to the drive assembly. The first switch is manually movable from an open position to a closed position. The automatic retraction switch is supported within the cavity and is coupled to the drive assembly. The automatic retraction switch is movable between open and closed positions. The automatic retraction switch control mechanism includes an actuator supported on the inner housing adjacent the automatic retraction switch. The actuator is movable from a first position to a second position in response to movement of the rack from the retracted position to the advanced position to move the automatic retraction switch from the open position to the closed position. When the first switch is in the closed position and the automatic retraction switch is in the open position, the drive assembly moves the rack towards the advanced position, and when the first switch is in the closed position and the automatic retraction switch is in the closed position, the drive assembly moves the rack towards the retracted position.

In aspects of the disclosure, the automatic retraction switch control mechanism includes a cap member that is supported on the actuator and engages the inner housing to resist movement of the actuator.

In some aspects of the disclosure, the cap member includes a slip resistant surface that is engaged with the inner housing.

In certain aspects of the disclosure, the slip-resistant surface of the cap member includes protrusions.

In aspects of the disclosure, the automatic retraction control mechanism includes a biasing member positioned to urge the cap member towards the inner housing.

In some aspects of the disclosure, the actuator defines a bore, and the biasing member is received in the bore to urge the cap member into an outer surface of the inner housing.

In certain aspects of the disclosure, the rack defines an elongate slot, and the actuator includes a finger that is received within the elongate slot.

In aspects of the disclosure, the rack includes a first engagement surface that is positioned at a proximal end of the elongate slot and engages the finger of the actuator when the rack is in the advanced position to move the actuator from the first position to the second position.

In some aspects of the disclosure, the rack includes a second engagement surface that is positioned at a distal end of the elongate slot and engages the finger of the actuator when the rack is in the retracted position to move the actuator from the second position to the first position.

In certain aspects of the disclosure, the actuator of the automatic retraction switch control mechanism is rotatably supported on the inner housing.

In aspects of the disclosure, the handle assembly includes a retraction button supported on the housing that is on engaged with the actuator and movable to move the actuator from the first position to the second position.

Other aspects of the disclosure are directed to a surgical device a tool assembly, an elongate body, and a powered handle assembly. The elongate body has a proximal portion and a distal portion. The tool assembly is supported on the distal portion of the elongate body and includes a first jaw and a second jaw that are movable between unclamped and clamped positions. The powered handle assembly is secured to the proximal portion of the elongate body and includes an outer housing defining a cavity. an inner housing secured within the cavity, a rack, a drive assembly, a first switch, an automatic retraction switch, and an automatic retraction switch control mechanism. The inner housing defines a longitudinally extending path through the inner housing. The rack is positioned along the longitudinally extending path of the inner housing and includes a first engagement surface. The rack is movable between retracted and advanced positions. The drive assembly is supported by the inner housing and is engaged with the rack to move the rack between the retracted and advanced positions. The first switch is supported within the cavity and is coupled to the drive assembly. The first switch is manually movable from an open position to a closed position. The automatic retraction switch is supported within the cavity and is coupled to the drive assembly. The automatic retraction switch is movable between open and closed positions. The automatic retraction switch control mechanism includes an actuator supported on the inner housing adjacent the automatic retraction switch. The actuator is movable from a first position to a second position in response to movement of the rack from the retracted position to the advanced position to move the automatic retraction switch from the open position to the closed position. When the first switch is in the closed position and the automatic retraction switch is in the open position, the drive assembly moves the rack towards the advanced position, and when the first switch is in the closed position and the automatic retraction switch is in the closed position, the drive assembly moves the rack towards the retracted position.

Other features of the disclosure will be appreciated from the following description.

### BRIEF DESCRIPTION OF THE DRAWINGS

Various aspects of the disclosed surgical device are described herein below with reference to the drawings, wherein:

FIG. 1 is a side perspective view of a surgical device according to aspects of the disclosure in an unclamped, pre-fired position;

FIG. 2 is an enlarged view of the indicated area of detail shown in FIG. 1 with an outer housing of a handle assembly of the surgical device shown in phantom and an articulation knob and shaft of an adapter assembly of the stapling device removed;

FIG. 2A is a schematic diagram of the switches and controller of the surgical device shown in FIG. 1;

FIG. 3 is an exploded side perspective view of components of the handle assembly shown in FIG. 2 including an inner housing, a rack, an automatic retraction switch, and a switch control mechanism;

FIG. 4 is side perspective view of an actuator of the switch control mechanism shown in FIG. 3;

FIG. 5 is a side perspective view of a cap of the switch control mechanism shown in FIG. 2;

FIG. 6 is a side perspective view of the rack of the handle assembly shown in FIG. 3;

FIG. 7 is a side perspective view from a first side of the internal components of the handle assembly shown in FIG. 3 as the actuator is keyed onto a first half of the inner housing of the handle assembly;

FIG. 8 is a side perspective view from a second side of the internal components of the handle assembly shown in FIG. 3 as the actuator is keyed onto the first half of the inner housing of the handle assembly;

FIG. 9 side perspective view from the first side of the internal components of the handle assembly shown in FIG. 3 as the actuator of the switch control mechanism is rotated into place on the first half of the inner housing of the handle assembly;

FIG. 10 side perspective view from the second side of the internal components of the handle assembly shown in FIG. 3 as the actuator of the switch control mechanism rotated into place on the first half of the inner housing of the handle assembly;

FIG. 11 is a side perspective view of the internal components of the handle assembly shown in FIG. 2 with the stapling device in a fully retracted position;

FIG. 12 is a cross-sectional view taken along section line 12-12 of FIG. 11;

FIG. 13 is a cross-sectional view taken along section line 13-13 of FIG. 11;

FIG. 14 is a cross-sectional view taken along section line 14-14 of FIG. 11;

FIG. 15 is a side cross-sectional cutaway view of the internal components of the handle assembly shown in FIG. 11 with the actuator shown in phantom as the rack moves from a retracted position towards an advanced position;

FIG. 16 is a side cross-sectional cutaway view of the internal components of the handle assembly shown in FIG. 11 with the actuator shown in phantom and the rack in the advanced position;

FIG. 17 is a side cross-sectional cutaway view of the internal components of the handle assembly shown in FIG. 11 with the actuator shown in phantom as the rack moves from the advanced position back towards the retracted position;

FIG. 18 is a side perspective view of an alternate version of the handle assembly of the stapling device shown in FIG. 1 with an outer housing section removed and including a retraction button assembly;

FIG. 19 is a side perspective view of an outer housing section and the retraction button assembly of the handle assembly shown in FIG. 18;

FIG. 20 is a side perspective view of the actuator of the switch control mechanism of the handle assembly shown in FIG. 18;

FIG. 21 is a side perspective view of another alternate version of the handle assembly of the stapling device shown in FIG. 1 with the outer housing of the handle assembly removed;

FIG. 22 is a side perspective view of a rack of the handle assembly shown in FIG. 21;

FIG. 23 is a perspective view from one side of an actuator of a switch control mechanism of the handle assembly shown in FIG. 21;

FIG. 24 is a perspective view from the other side of the actuator shown in FIG. 23;

FIG. 25 is a cross-sectional view taken along section line 25-25 of FIG. 21;

FIG. 26 is a cross-sectional view taken along section line 26-26 of FIG. 25 with the handle assembly with the handle assembly in the fired position; and

FIG. 27 a cross-sectional view taken along section line 26-26 of FIG. 25 with the handle assembly with the handle assembly in the retracted position.

### DETAILED DESCRIPTION

The disclosed surgical device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, and similar terms are used to assist in understanding the description and are not intended to limit the disclosure.

In this description, the term “proximal” is used generally to refer to that portion of the device that is closer to a clinician, while the term “distal” is used generally to refer to that portion of the device that is farther from the clinician. In addition, the term “endoscopic” is used generally to refer to endoscopic, laparoscopic, arthroscopic, and/or any other procedure conducted through a small diameter incision or cannula. Further, the term “clinician” is used generally to refer to medical personnel including doctors, nurses, surgeons, and support personnel.

This disclosure is directed to a powered surgical device that includes a handle assembly having a first switch for initiating clamping and firing of the surgical device and a second switch for initiating automatic retraction. The second switch is controlled by a switch control mechanism that is engaged with a rack in the handle assembly to initiate automatic retraction when firing is completed.

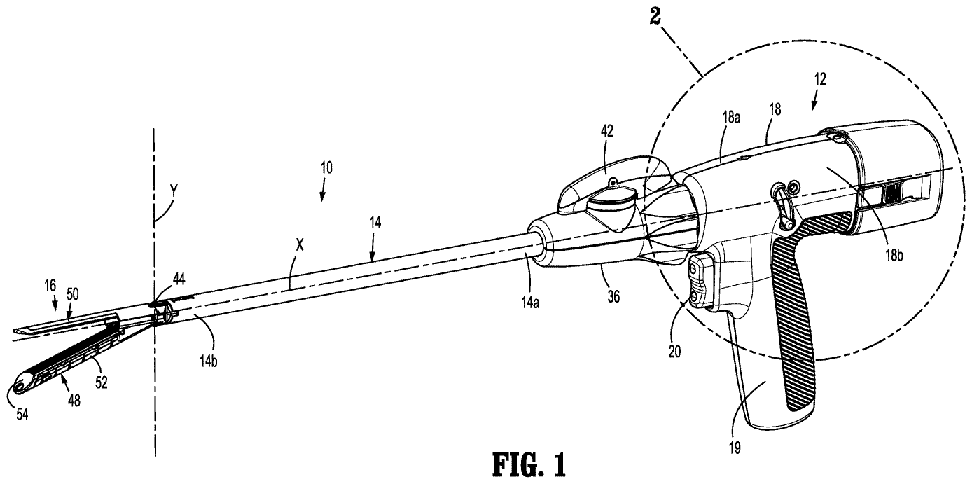

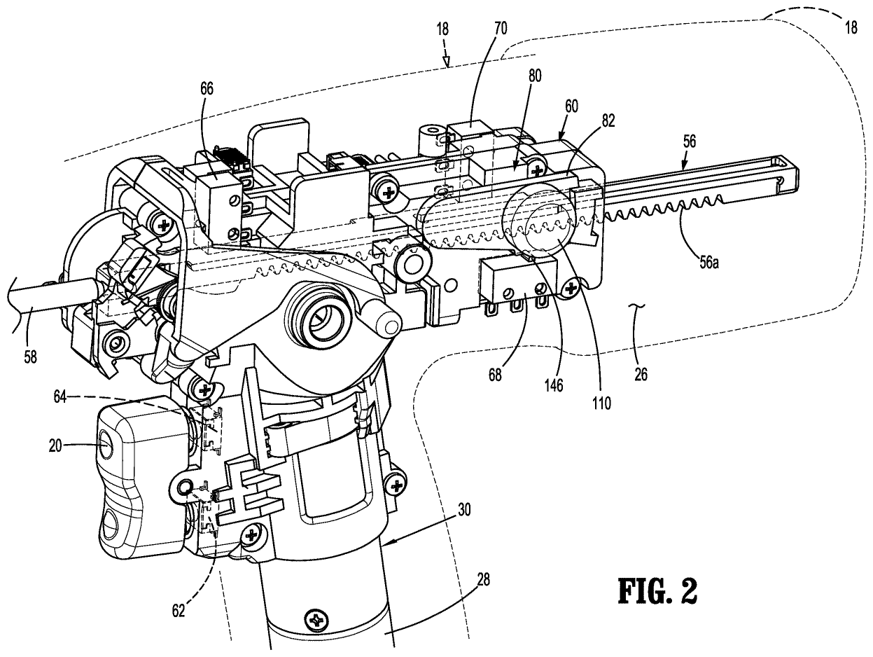

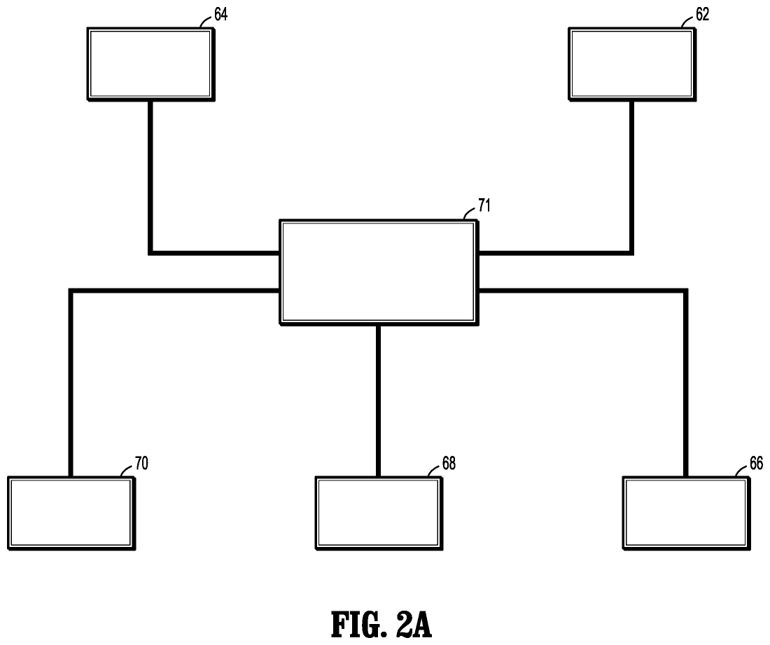

FIGS. 1 and 2 illustrate a surgical device shown generally as stapling device 10 which includes a powered handle assembly 12, an elongate body or adapter assembly 14, and a tool assembly 16. The handle assembly 12 includes an outer housing 18 that forms a stationary handle portion 18a. The stationary handle portion 18 supports an actuation button or buttons, e.g., a rocker 20, that can be actuated to control operation of the various functions of the stapling device 10, e.g., clamping and firing of the stapling device 10. In aspects of the disclosure, the outer housing 18 of the handle assembly 12 is formed from half-sections 22 and 24 that are secured together to define a cavity 26 (FIG. 2) that encloses internal components of the surgical device 10. In aspects of the disclosure, the stationary handle portion 18a of the outer housing 18 supports batteries 28 and a drive assembly, e.g., a motor/gear assembly 30.

The elongate body 14 of the stapling device 10 has a proximal portion 14a and a distal portion 14b and defines a longitudinal axis “X”. The proximal portion 14a of the elongate body 14 supports a rotation knob 36 and an articulation lever 42 that is supported on the rotation knob 36. The rotation knob 36 is coupled to the handle assembly 12 and supports the elongate body 14 to facilitate rotation of the elongate body 14 and the tool assembly 16 about the longitudinal axis “X” in relation to the handle assembly 12.

The tool assembly 16 is secured to the distal portion 14b of the elongate body 14 by a pivot member 44 that defines an axis “Y” that is transverse to the longitudinal axis “X”. The articulation lever 42 is operatively coupled to the tool assembly 16 via an articulation linkage (not shown) such that manipulation of the articulation lever 42 causes articulation of the tool assembly 16 about the axis “Y” between a non-articulated position in which the tool assembly 16 is aligned with the longitudinal axis “Y” and articulated positions in which a longitudinal axis of the tool assembly 16 and the longitudinal axis “X” of the elongate body 14 define acute angles.

The tool assembly 16 includes a first jaw and a second jaw. In aspects of the disclosure, the first jaw supports a cartridge assembly 48 and the second jaw supports an anvil assembly 50. The cartridge assembly 48 includes a channel 52 that supports a staple cartridge 54. In aspects of the disclosure, the staple cartridge 54 is removably received in the channel 52 and can be replaced after each firing of the stapling device 10 to facilitate reuse of the stapling device 10. The channel 52 is pivotably coupled to the anvil assembly 50 and is movable in relation to the anvil assembly 50 between unclamped and clamped positions. Alternately, the anvil assembly 50 may be pivotably coupled to the cartridge assembly 48 and movable in relation to the cartridge assembly 48 between unclamped and clamped positions.

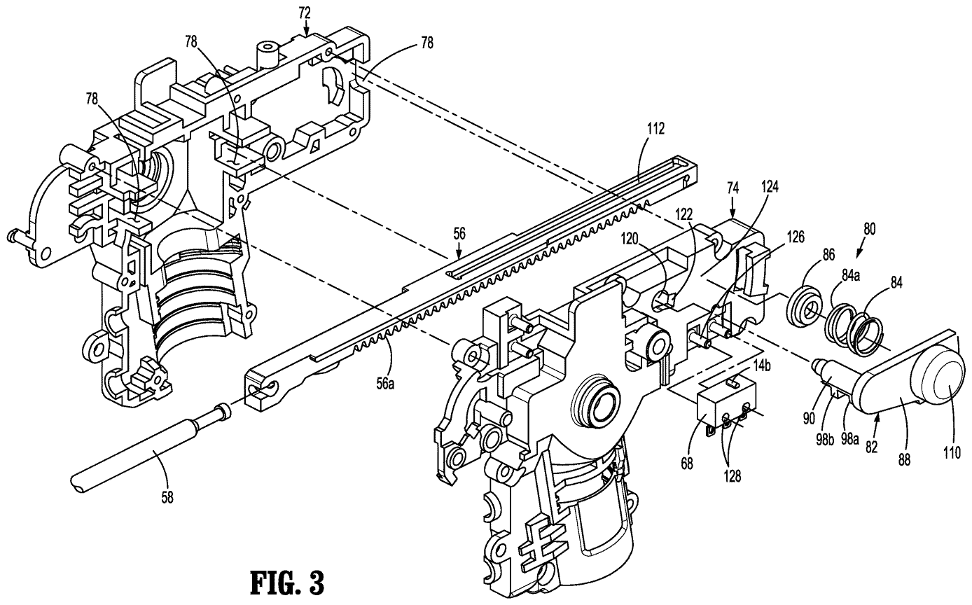

FIGS. 2 and 3 illustrate internal components of the handle assembly 12 which includes a rack 56, a control rod 58, an inner housing 60 (FIG. 2), a first switch 62, a second switch 64, a third switch 66, a fourth switch 68, a fifth switch 70, and a switch control mechanism 72. The inner housing 60 is formed from sections 72 and 74 and is secured within the outer housing 18 of the handle assembly 12 with screws or the like (not shown) to support and align the internal components of the handle assembly 12 (FIG. 1) within the outer housing 18.

The inner housing 60 defines a longitudinally extending path or channel 78 (FIG. 3) that extends from a proximal end of the inner housing 60 to the distal end of the inner housing 60 and receives and supports the rack 56 for linear movement between rack retracted and advanced positions. The control rod 58 has a proximal end that is coupled to the distal portion of the rack 56 and is movable between rod retracted and advanced positions in response to movement of the rack 56 between the rack advanced and retracted positions. The control rod 58 is coupled to a drive assembly (not shown) that is movable in response to movement of the rack 56 between the rack retracted and rack advanced positions within the tool assembly 16 to actuate the tool assembly 12, i.e., move the tool assembly 12 between the unclamped and clamped positions and fire the stapling device. The rack 56 includes teeth 56a that are engaged with the motor/gear assembly 30 such that activation of the motor/gear assembly 30 moves the rack 56 between the rack retracted and rack advanced positions within the channel 72 of the inner housing 60. The inner housing 60 supports the motor/gear assembly 30 and the rack 56 to maintain proper alignment between the components.

The switches 62-70 of the handle assembly 12 are connected to logical gate circuits to input signals into a motor driver to control forward and reverse motion of a motor of the motor/gear assembly 30. In aspects of the disclosure, the motor of the motor/gear assembly 30 includes a DC brushed motor although other types of motors are envisioned. The switches 62-70 are movable between open and closed positions to provide signals to a controller 71 (FIG. 2A) to control movement and the direction of movement of the motor/gear assembly 30. The signals provide status information to the controller regarding position of the rack 56 and other mechanical components of the stapling device 10. In some aspects of the disclosure the switches may be coupled to the controller via logical gate circuits. In aspects of the disclosure, the first and second switches 62 and 64 are embedded in a printed circuit board assembly (PCBA) (not shown) and are normally open. The first switch 62 may be manually actuated via the rocker 20 (FIG. 1) to a closed position to advance the rack 56 and move the tool assembly to the clamped position, and to advance the rack 56 and fire the stapling device 10 (FIG. 1). The second switch 64 may be manually actuated via the rocker 20 to retract the rack 56 and move the tool assembly 16 from the clamped position to the unclamped position, and to retract the rack 56 and move the tool assembly 16 from the fired position to the unclamped position.

The third switch 66 is normally in the open position and is automatically moved to the closed position when the tool assembly 16 is moved from the unclamped position to the clamped position to stop advancement of the rack 56 after clamping is complete. The fourth switch 68 is provided to stop advancement of the rack 56 when firing is complete and to stop retraction of the rack 56 when the rack 56 reaches the rack retracted position. The fourth switch can also trigger automatic retraction of the rack 56 when the first switch 62 is released and the second switch 64 is in the open position. Switch 5 is provided to end retraction of the rack 56 when the second switch 64 is in the held in the closed position via the rocker 20 (FIG. 1) and the rack 56 reaches the rack retracted position.

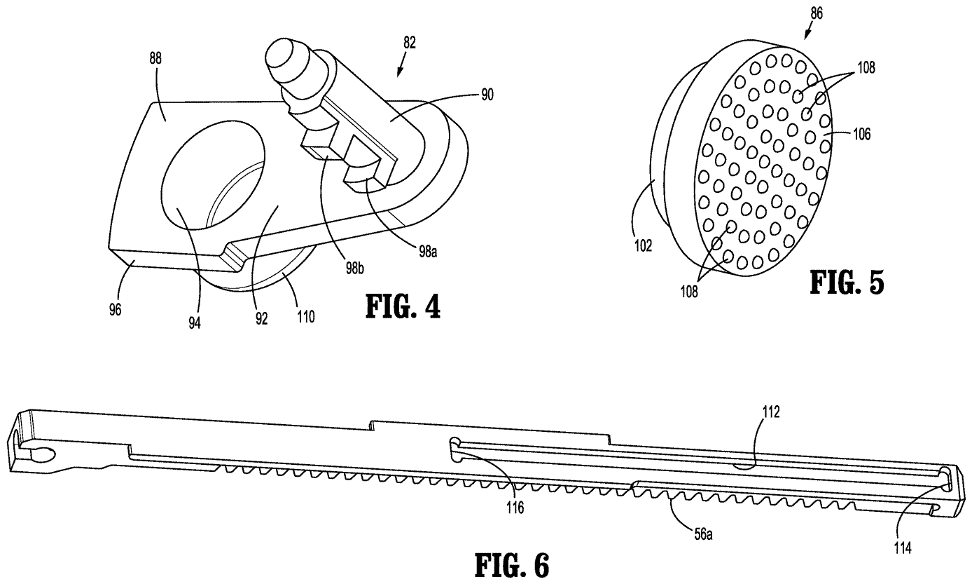

FIG. 3 illustrates a switch control mechanism 80 for controlling operation of the fourth switch 68. The switch control mechanism 80 includes an actuator 82, a biasing member 84, and a cap member 86. The actuator 82 also shown in FIG. 4 includes a body 88 and a post 90 that extends transversely from the body 88. The body 88 has an inner surface 92 and defines a transverse bore 94 that opens onto the inner surface 92 of the body 88 of the actuator 82. In aspects of the disclosure, the body 88 of the actuator includes a bottom surface that defines an abutment member 96. The post 90 extends inwardly from the inner surface 92 of the actuator 82 and includes two downwardly extending fingers 98a and 98b.

FIG. 5 illustrates the cap member 86 of the switch control mechanism 80 which includes a stepped body 100 that defines an inwardly extending annular extension 102 and a cap portion 104. The cap portion 104 has an inwardly facing slip-resistant surface 106. In aspects of the disclosure, the slip-resistant surface 106 includes a plurality of protrusions 108. The annular extension 102 is received in the transverse bore 94 of the actuator 82 such that the cap member 86 is movable transversely in relation to the body 88 of the actuator 82.

The biasing member 84 of the switch control mechanism 80 is received in the transverse bore 94 of the actuator 82 and is positioned to urge the cap member 86 inwardly towards the section 74 of the inner housing 60 of the handle assembly 12 as described in further detail below. In aspects of the disclosure, the outer end of the transverse bore 94 of the actuator 82 is closed by a cover 110 and the biasing member 84 includes a coil spring that is compressed between the cover 110 and the cap member 86.

FIG. 6 illustrates the rack 56 which includes the teeth 56a and defines an elongate slot 112. The elongate slot 112 has a proximal end defined by a first engagement surface 114 and a distal end defined by a second engagement surface 116. The elongate slot 112 receives the downwardly extending finger 98b of the actuator 82 of the switch control mechanism when the switch control mechanism 80 is coupled to the inner housing 60 (FIG. 2) as described in further detail below.

The actuator 82 is rotatably supported on the section 74 of inner housing 60 about an axis defined by the post 90 that is transverse to the longitudinal axis “X” of the elongate body 14 (FIG. 1). The half-section 90 defines a circular opening 120 that has a notch 122 that receives the fingers 98a and 98b of the post 90 when the post is inserted through the opening 120. The half-section 90 also has a flat outer surface 124 (FIG. 3) that engages the inner surface 92 of the actuator 82 when the actuator 82 is mounted on the section 74.

The fourth switch 68 is mounted to the outer surface of the section 74 of the inner housing 60 at a position below the flat outer surface 124 of the section 74 of the inner housing 60. In aspects of the disclosure, the section 74 of the inner housing 60 includes posts 126 that are received within bores 128 defined in the fourth switch 68 to mount the fourth switch 68 to the inner housing 60. Alternately, other mounting techniques or members may be used to mount the fourth switch 68 to the inner housing 60.

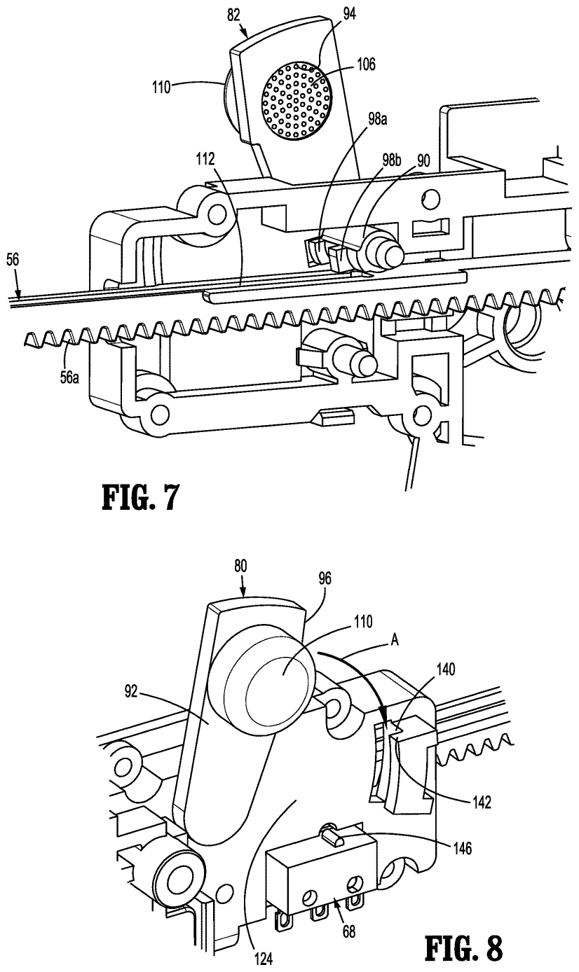

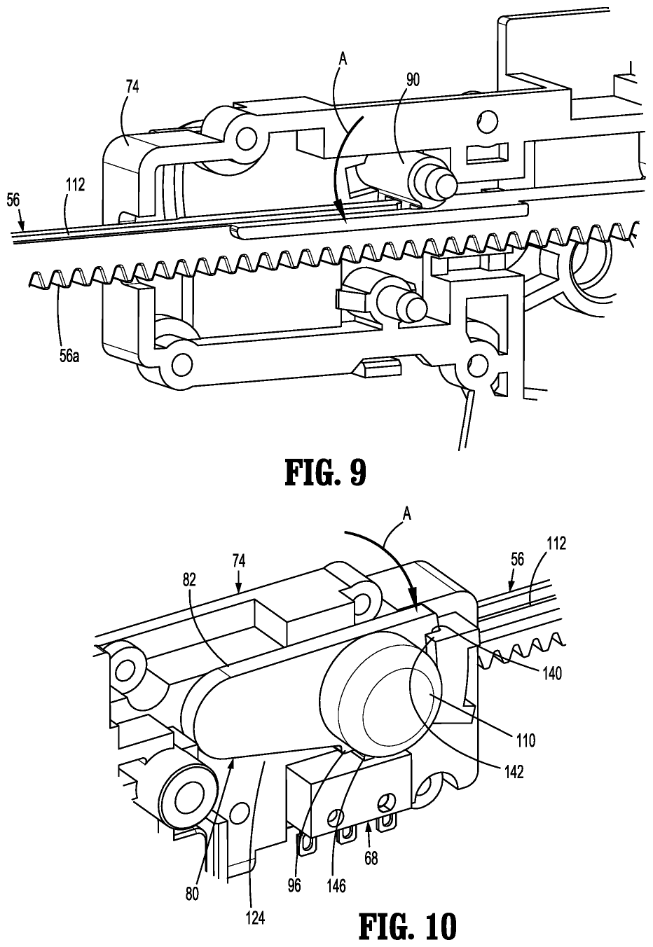

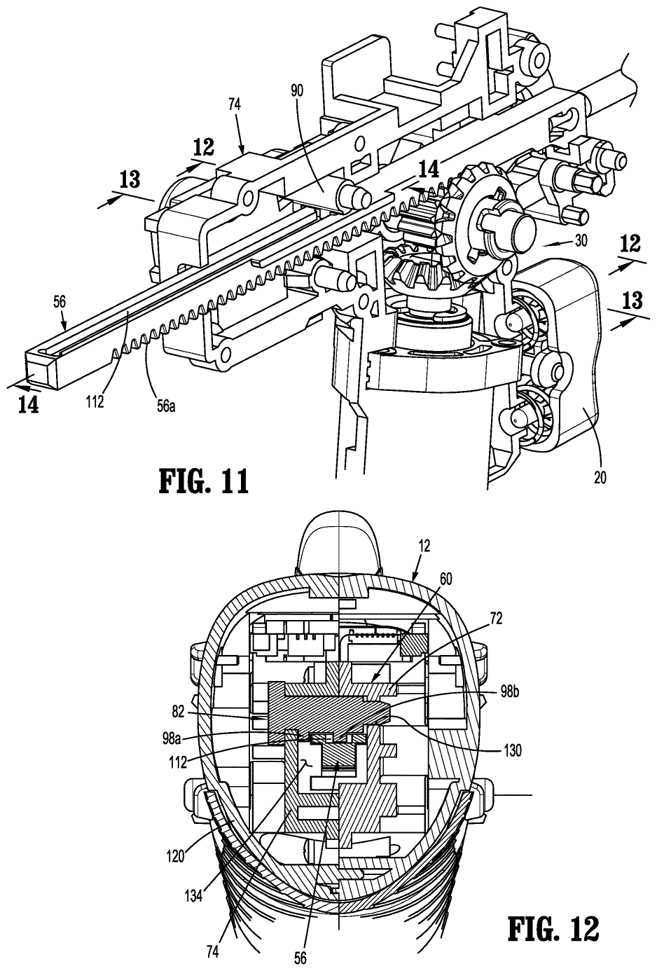

FIGS. 8-13 illustrate the switch control mechanism 80 as the switch control mechanism 80 is mounted onto the inner housing 60 of the handle assembly 12 (FIG. 1). When the switch control mechanism 80 is mounted onto the section 74 of the inner housing 60, the post 90 of the actuator 82 is inserted through the opening 120. To insert the post 90 fully through the opening 120, the fingers 98a, 98b of the post 90 of the actuator 82 must be aligned with the notch 122 (FIG. 3) of the opening 120 as the post 90 is inserted through the opening 120. When the post 90 is fully inserted into the opening 120, an end of the post 90 spaced from the body 88 of the actuator 82 is received in an opening 130 (FIG. 12) in the section 72 of the inner housing 60 to rotatably support the actuator 82 on the inner housing 60, and the fingers 98a, 98b are positioned in a cavity 134 (FIG. 12) defined between sections 72, 74 of the inner housing 60. It is noted that when the post 90 is inserted into the opening 120 of the inner housing 60 (FIG. 7), the biasing member 84 and cap member 86 of the switch control mechanism 80 are received within the transverse bore 94 of the actuator 82 such that the slip-resistant surface 106 of the actuator 82 faces inwardly towards the flat outer surface 124 of the inner housing 60.

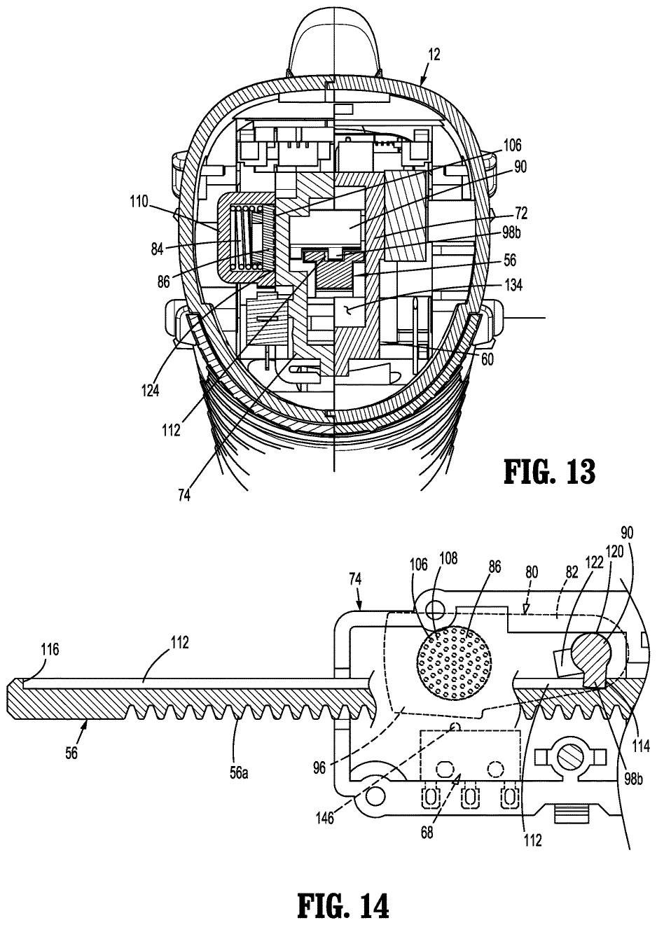

Once the post 90 is fully inserted into the opening 120 in the inner housing 60, the actuator 82 can be rotated in the direction of arrows “A” in FIGS. 8-10 to position the slip-resistant surface 106 of the actuator 82 into engagement with the flat outer surface 124 of the inner housing 60. As the actuator 82 is rotated in the direction of arrow “A”, the proximal end of the body 88 is received within a channel 140 (FIG. 10) defined in the section 74 of the inner housing 60. The channel 140 is defined in part by an over-hang 142 that prevents outward movement of the actuator 82 in relation to the inner housing 60 when the proximal end of the body 88 is received within the channel 140. As the actuator 82 is rotated into the channel 140, the finger 98a of the actuator 82 moves to a position between the rack 56 and the inner housing 60, and the finger 98b moves into the slot 112 of the rack 56 (FIG. 12). In this position, the overhang 142 (FIG. 10) of the inner housing 60 prevents the post 90 of the actuator 82 of the switch control mechanism 80 from being removed from the opening 120 of the inner housing 60. In addition, the biasing member 84 of the switch control mechanism 80 urges the cap member 86 from the transverse bore 94 defined in the actuator 82 into the flat outer surface 124 of the inner housing 60 (FIG. 13) to obstruct rotation of the actuator 82 in a direction opposite to arrow “A”. More specifically, the protrusions 108 of the cap member 86 are biased into engagement with the flat outer surface 124 of the inner housing 60 to create friction between the switch control mechanism 80 and the inner housing 60 to maintain the position of the actuator 82 in relation to the inner housing 60 during movement of the rack 56 (FIG. 13). It is noted that the switch control mechanism 80 will function without the cap member 86. More specifically, the cap member 86 can be removed such that the biasing member 84 of the switch control mechanism 80 is in direct contact with the inner housing 60 to maintain the position of the actuator 82 in relation to the inner housing 60 during movement of the rack 56. In aspects of the disclosure, the biasing member 84 may be in the form of a coil spring that includes a flat surface 84a (FIG. 3) at one end of the biasing member 84 that is engaged with the inner housing 60. In the assembled condition, the abutment member 96 of the actuator 82 is positioned above a switch button 146 such that the fourth switch 68 is in the open position.

FIG. 14 illustrates the switch control mechanism 80 and the rack 56 with the rack 56 in a retracted position. This is the position the switch control mechanism 80 and the rack 56 are in when the stapling device 10 (FIG. 1) is as shown in FIG. 1 in an unclamped position. When the rack 56 is in a retracted position, the finger 98b extending from the post 90 is positioned in the proximal end of the elongate slot 112 of the rack 56 adjacent to the first engagement surface 114 of the rack 56.

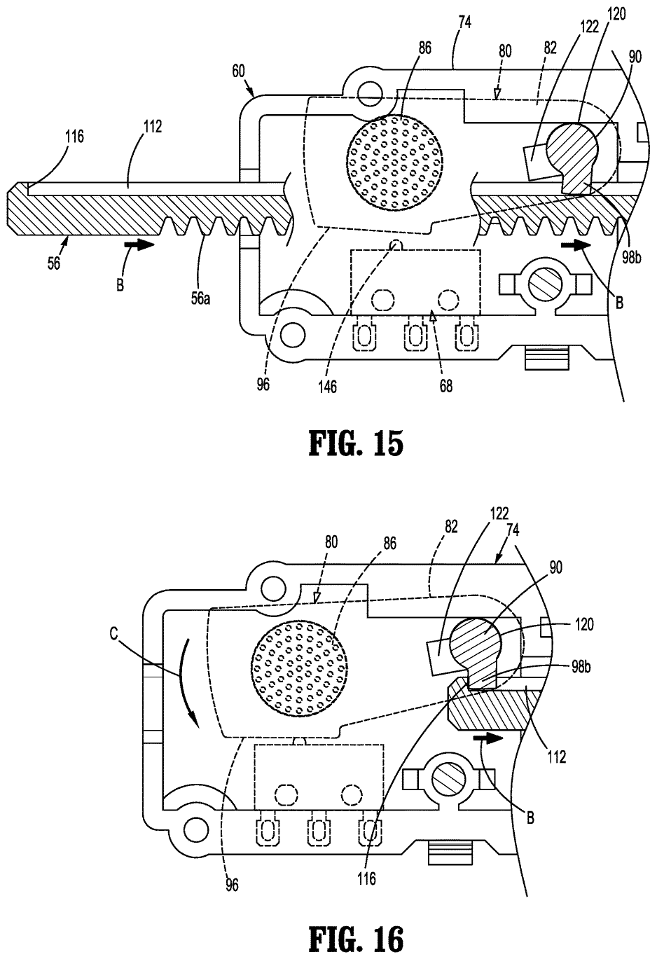

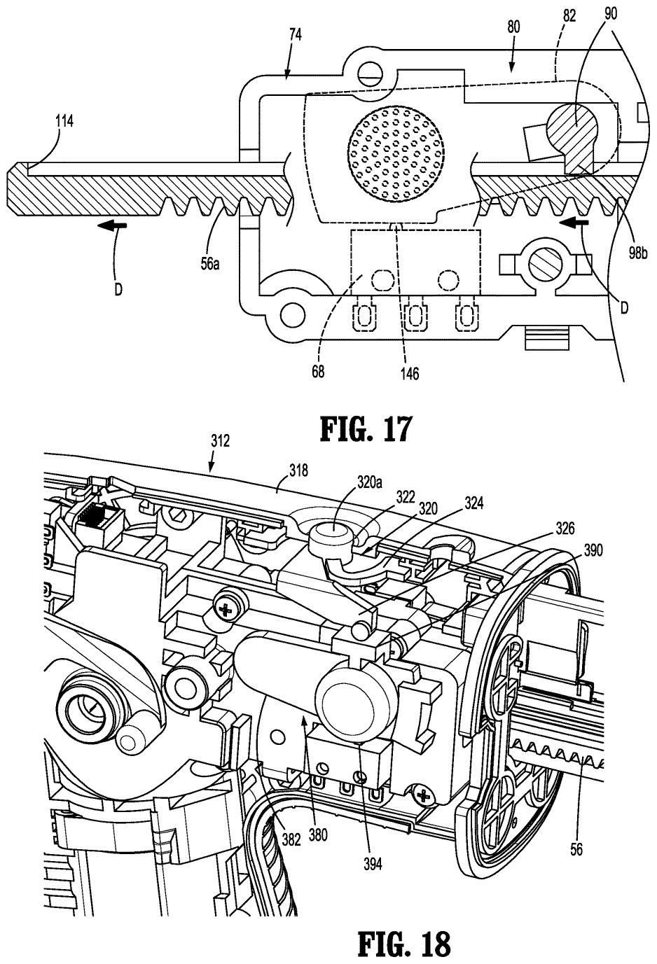

FIGS. 15-17 illustrate the rack 56 as the rack 56 is moved from the retracted position to the advanced position through the inner housing 60 of the handle assembly 12 (FIG. 1) in the direction of arrows “B”. As described briefly above, the rack can be advanced to clamp and fire the stapling device 10 by pressing the rocker 20 manually (FIG. 1) to close the first switch 62 (FIG. 2). The rocker 20 must be held in the position to close the first switch 62 during both clamping and subsequently during firing of the stapling device 10 (FIG. 1) in two separate actions. After clamping is completed and the rocker 20 is pressed a second time and held by the clinician to close the first switch 62 and advance the rack 56 to the advanced position, the finger 98b will travel through the elongate slot 112 of the rack 56 until the finger 98b is engaged by the second engagement surface 116 of the rack 56. When this happens, continued movement of the rack 56 will rotate the switch control mechanism 80 in the direction of arrow “C” in FIG. 16 such that the abutment member 96 of the actuator 82 of the switch control mechanism 80 engages the switch button 146 to move the fourth switch to the closed position. When this occurs with the first switch 62 (FIG. 2) held in the closed position, the direction of rotation of the motor gear assembly 30 (FIG. 2) will reverse to automatically retract the rack 56 in the direction of arrow “D” in FIG. 17. It is noted that when the rack 56 is fully retracted and the first engagement surface 114 of the rack 56 engages the finger 98b of the actuator 82, the actuator 82 will be rotated back to the position shown in FIG. 14 such that the fourth switch 68 returns to the open position to stop retraction of the rack 56.

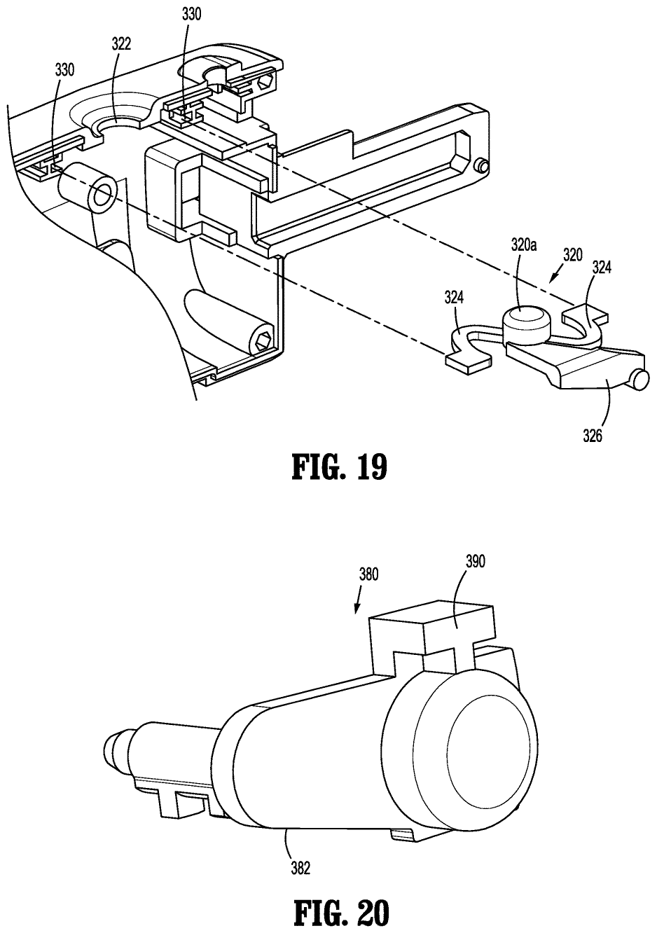

FIGS. 18-20 illustrate an alternate version of the handle assembly 12 (FIG. 1) shown generally as handle assembly 312. Handle assembly 312 is identical to handle assembly 12 except that the handle assembly 312 also includes a retraction button assembly 320. The retraction button assembly 320 is accessible through an opening 322 in a housing 318 of the handle assembly 312 and includes a retraction button 320a, a biasing portion 324, and an engaging portion 326. The biasing portion 324 is secured to the retraction button 320a and to the housing 318 and is configured to move the retraction button 320 upwardly within the opening 322 of the housing 318. In aspects of the disclosure, the biasing portion 324 includes elastic arms that are received within slots 330 of the housing 318 and are in tension to urge the retraction button 320a upwardly through the opening 322 of the housing 318. The engaging portion 326 of the retraction button assembly 320 is secured to the retraction button 320a and is supported on an upper surface of the actuator 382 of the switch control mechanism 380. The actuator 382 is identical to the actuator 380 described above except that the actuator 382 includes a platform 390 that supports the engaging portion 326 of the retraction button assembly 320.

The handle assembly 312 functions identically to the handle assembly 12 described above except that a clinician can initiate automatic retraction at any position of the rack 356 between the retracted and advanced positions when the first switch 62 (FIG. 2) is in a closed position. More specifically, prior to movement of the rack to the fully advanced position with the first switch 62 held in the closed position with the rocker 20 (FIG. 1), a clinician can depress the retraction button 320a to move the engaging portion 326 downwardly into the platform 390 of the actuator 382 to close the fourth switch 68. More specifically, when the retraction button 320a is depressed by the clinician, the engaging portion 326 of the actuator 382 moves downwardly against the urging of the biasing portion 324 to press against the platform 390 of the actuator 382. When this happens, the actuator 382 pivots downwardly into engagement with the switch button 394 of the fourth 368 to close the fourth switch 68.

In aspects of the disclosure, the retraction button assembly 320 can be integrally formed. For example, the biasing portion 324 can be over molded onto the button 320a and the engaging portion 326 to form an integral assembly 320. Other integral and multi-piece configurations are envisioned.

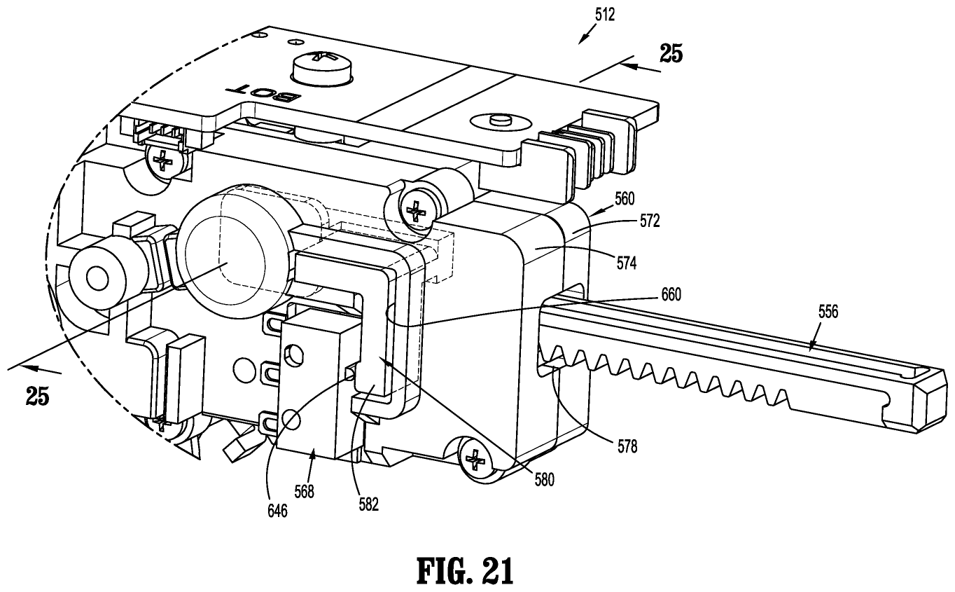

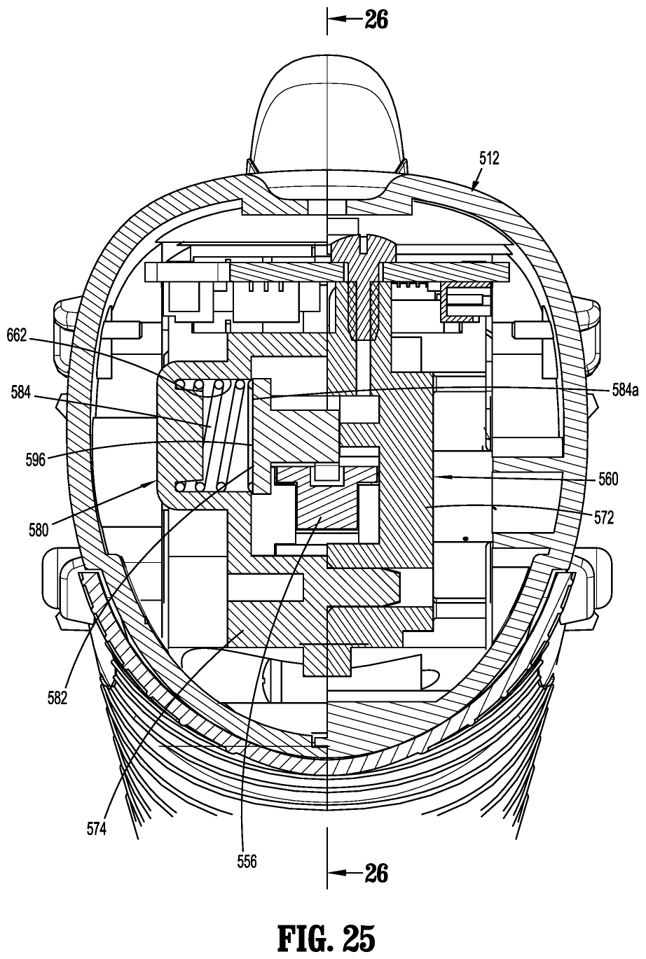

FIGS. 21-27 illustrate another alternate version of the handle assemblies 12 and 312 shown generally as handle assembly 512. The handle assembly 512 is like the handle assemblies 12 and 312 and includes a rack 556, an inner housing 560, switches including a fourth switch 568, and a switch control mechanism 580 including an actuator 582 and a biasing member 584 (FIG. 25). The inner housing 560 is formed from sections 572 and 574 that define a longitudinally path or channel 578 (FIG. 21) that extends through the inner housing 560 and receives the rack 556. The rack 556 is movable within the channel 578 between retracted and advanced positions. As described above regarding the rack 56 (FIG. 3), the rack 556 has a distal end that is coupled to a control rod 558 (FIG. 27) that is coupled to a drive assembly (not shown) that is movable to actuate the tool assembly 16 (FIG. 1).

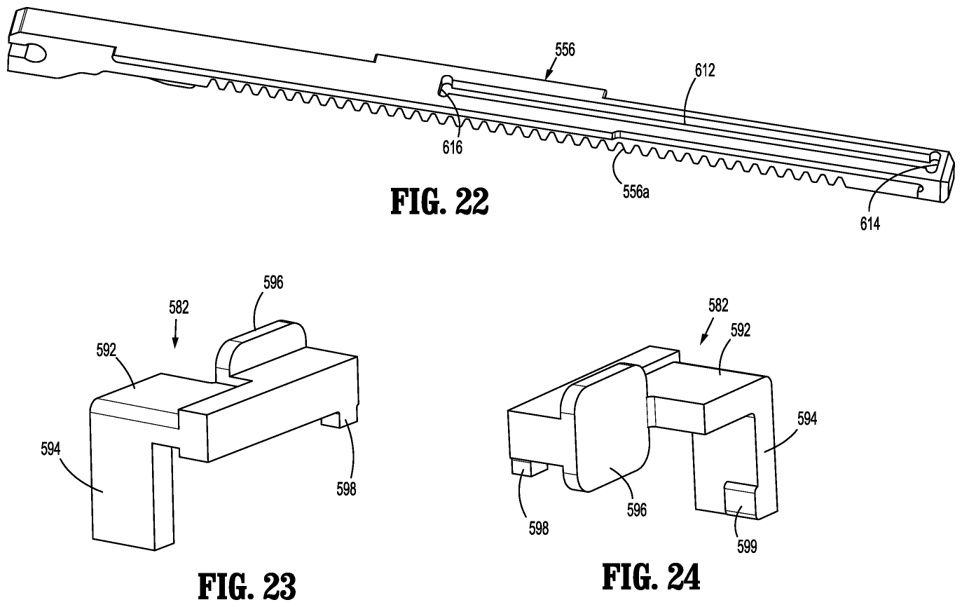

FIG. 22 illustrates the rack 556 which includes teeth 556a and defines an elongate slot 612. The elongate slot 612 has a proximal end defined by a first engagement surface 614 and a distal end defined by a second engagement surface 616. The elongate slot 612 receives a downwardly extending finger 598 of the actuator 582 of the switch control mechanism 580 when the switch control mechanism 580 is coupled to the inner housing 560 (FIG. 2) as described in further detail below.

FIGS. 23 and 24 illustrate the actuator 582 of the switch control mechanism 580 which includes a body 592 having a downwardly extending leg 594, a biasing member engagement surface 596, and the downwardly extending finger 598. The body 592 is supported on top of the rack 556 such that the finger 598 is received within the elongate slot 612 of the rack 556. The downwardly extending leg 594 is positioned outwardly of the finger 598 and extends along a proximal side of the fourth switch 568 (FIG. 21). The downwardly extending leg 594 of the actuator 582 has an abutment member 599 (FIG. 24) that is positioned adjacent to the switch button 646 of the fourth switch 568. The abutment member 599 is movable in relation to the fourth switch 569 as described in further detail below to move the switch button 646 from an open position to a closed position.

The actuator 582 is supported on the housing 560 for movement between retracted and advanced positions in response to movement of the rack 556 between the rack retracted and rack advanced positions. In aspects of the disclosure, the section 574 of the inner housing 560 defines a cavity 660 (FIG. 21) on an outer surface of the inner housing 560 that receives a portion of the body 592 of the actuator 582. When the rack 556 moves from the rack retracted position to the rack advanced position, the first engagement surface 614 of the rack 556 engages the finger 598 of the actuator 582 of the switch control mechanism 580 to move the actuator 582 from the retracted position to the advanced position. When the actuator 582 is in the advanced position, the abutment member 599 on the downwardly extending leg 594 of the actuator 582 engages the switch button 646 of the fourth switch 568 to move the switch 568 from the open position to the closed position. Similarly, when the rack 556 moves from the rack advanced position to the rack retracted position, the second engagement surface 616 engages the finger 598 to move the actuator 582 from the advanced position to the retracted position to allow the switch 568 to return to the open position.

FIG. 25 illustrates the switch control mechanism 580 including the actuator 582 and the biasing member 584. In aspects of the disclosure, the biasing member 584 of the switch control mechanism 580 is received within a blind bore 662 (FIG. 25) defined by the section 574 of the inner housing 560. The biasing member 584 is in compression and presses against the biasing member engagement surface 596 of the actuator 582 to press the actuator 582 against an inner surface of the inner housing 560. The biasing member 584 functions in a similar manner to the biasing member 84 (FIG. 3). More specifically, the biasing member 584 urges the actuator 582 against the inner surface of the inner housing 560 to resist movement of the actuator 582 between the retracted and advanced positions.

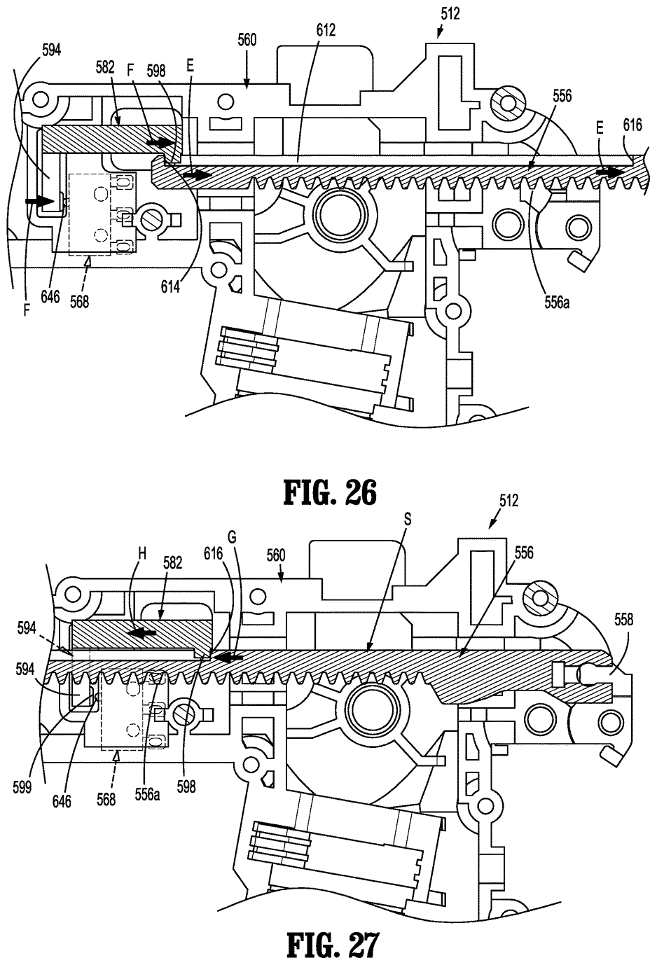

FIG. 26 illustrates the handle assembly 512 as the rack 556 moves in the direction of arrows “E” from the retracted position to the advanced position to fire the stapling device 10 (FIG. 1). As the rack 556 moves from the rack retracted position to the rack advanced position, the first engagement surface 614 of the rack 556 engages the finger 598 of the actuator 582 of the switch control mechanism 580 to move the actuator 582 in the direction of arrows “F” from the retracted position to the advanced position and move the fourth switch 568 from the open position to the close position. When this occurs, the rack 556 will automatically retract as described above regarding the handle assembly 12. It is noted that the actuator 582 only needs to move a short distance from its retracted position to its advanced position to move the switch button 646 of the switch 568 from the open position to the closed position.

FIG. 27 illustrates the handle assembly 512 as the rack 556 moves in the direction of arrow “G” from the rack advanced position back to the rack retracted position after the stapling device 10 (FIG. 1) is fired. As the rack 556 moves from the advanced position back to the retracted position, the second engagement surface 616 of the rack 556 engages the finger 598 of the actuator 582 of the switch control mechanism 580 to move the actuator 582 in the direction of arrows “H” from its advanced position to its retracted position. When the actuator 582 moves from the advanced position back to the retracted position, the abutment member 599 on the downwardly extending leg 594 of the actuator 582 moves away from the switch button 646 to allow the fourth switch 568 to move from the closed position to the open position. When this occurs, the motor/gear assembly 30 is deactivated and the rack 556 stops retracting.

Operation of the fourth switch 568 as described above causes the stapling device 10 to function in the manner described above regarding the handle assembly 12 (FIG. 2). The switch control mechanism 580 allows the rack 556 to automatically retract after firing of the stapling device 10 (FIG. 1) is completed.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is

1. A powered handle assembly comprising:
an outer housing defining a cavity;
an inner housing secured within the cavity, the inner housing defining a longitudinally extending path through the inner housing;
a rack positioned along the longitudinally extending path of the inner housing, the rack including a first engagement surface and movable between retracted and advanced positions;
a drive assembly supported by the inner housing, the drive assembly engaged with the rack and operable to move the rack between the retracted and advanced positions;
a first switch supported within the cavity and coupled to the drive assembly, the first switch manually movable from an open position to a closed position;
an automatic retraction switch supported within the cavity, the automatic retraction switch coupled to the drive assembly and movable between open and closed positions; and
an automatic retraction switch control mechanism including an actuator supported on the inner housing adjacent the automatic retraction switch, the actuator movable from a first position to a second position in response to movement of the rack from the retracted position to the advanced position to move the automatic retraction switch from the open position to the closed position;
wherein the first switch and the automatic retraction switch are configured such that when the first switch is in the closed position and the automatic retraction switch is in the open position, the drive assembly is actuated to move the rack towards the advanced position, and when the first switch is in the closed position and the automatic retraction switch is in the closed position, the drive assembly is actuated to move the rack towards the retracted position.

2. The powered handle assembly of claim 1, wherein the automatic retraction switch control mechanism includes a cap member supported on the actuator, the cap member engaging the inner housing to resist movement of the actuator between the first and second positions.

3. The powered handle assembly of claim 2, wherein the cap member includes a slip resistant surface that is engaged with the inner housing.

4. The powered handle assembly of claim 1, wherein the actuator is movable longitudinally between retracted and advanced positions to move the automatic retraction switch between the open and closed positions.

5. The powered handle assembly of claim 2, wherein the automatic retraction control mechanism includes a biasing member positioned to urge the cap member into engagement with the inner housing.

6. The powered handle assembly of claim 1, wherein the automatic retraction control mechanism includes a biasing member and the actuator includes a surface that is engaged by the biasing member, the biasing member urging the actuator into engagement with the inner housing

7. The powered handle assembly of claim 1, wherein the rack defines an elongate slot, and the actuator includes a finger that is received within the elongate slot.

8. The powered handle assembly of claim 7, wherein the rack includes a first engagement surface positioned at a proximal end of the elongate slot, the first engagement surface engaging the finger of the actuator when the rack is in the advanced position to move the actuator from the first position to the second position.

9. The powered handle assembly of claim 8, wherein the rack includes a second engagement surface positioned at a distal end of the elongate slot, the second engagement surface engaging the finger of the actuator when the rack is in the retracted position to move the actuator from the second position to the first position.

10. The powered handle assembly of claim 1, further including a retraction button supported on the housing, the retraction button engaged with the actuator and movable to move the actuator from the first position to the second position.

11. A surgical device comprising:
an elongate body having a proximal portion and a distal portion;
a tool assembly supported on the distal portion of the elongate body, the tool assembly including a first jaw and a second jaw, the first and second jaws being movable between unclamped and clamped positions; and
a powered handle assembly secured to the proximal portion of the elongate body, the powered handle assembly including:
an outer housing defining a cavity;
an inner housing secured within the cavity, the inner housing defining a longitudinally extending path through the inner housing;
a rack positioned along the longitudinally extending path of the inner housing, the rack including a first engagement surface and movable between retracted and advanced positions;
a drive assembly supported by the inner housing, the motor gear/assembly engaged with the rack and operable to move the rack between the retracted and advanced positions;
a first switch supported within the cavity and coupled to the drive assembly, the first switch manually movable from an open position to a closed position;
an automatic retraction switch supported within the cavity, the automatic retraction switch coupled to the drive assembly and movable between open and closed positions; and
an automatic retraction switch control mechanism including an actuator supported on the inner housing adjacent the automatic retraction switch, the actuator movable from a first position to a second position in response to movement of the rack from the retracted position to the advanced position to move the automatic retraction switch from the open position to the closed position;
wherein the first switch and the automatic retraction switch are configured such that when the first switch is in the closed position and the automatic retraction switch is in the open position, the drive assembly is actuated to move the rack towards the advanced position, and when the first switch is in the closed position and the automatic retraction switch is in the closed position, the drive assembly is actuated to move the rack towards the retracted position.

12. The surgical device of claim 11, wherein the automatic retraction switch control mechanism includes a cap member supported on the actuator, the cap member engaging the inner housing to resist movement of the actuator.

13. The surgical device of claim 12, wherein the cap member includes a slip resistant surface that is engaged with the inner housing.

14. The surgical device of claim 13, wherein1, wherein the actuator is movable longitudinally between retracted and advanced positions to move the automatic retraction switch between the open and closed positions ..

15. The surgical device of claim 14, wherein the automatic retraction switch control mechanism includes a biasing member positioned to urge the cap member towards the inner housing.

16. The surgical device of claim 15, wherein the actuator defines a bore, and the biasing member is received in the bore to urge the cap member into an outer surface of the inner housing.

17. The surgical device of claim 11, wherein the rack defines an elongate slot, and the actuator includes a finger that is received within the elongate slot.

18. The surgical device of claim 17, wherein the rack includes a first engagement surface positioned at a proximal end of the elongate slot, the first engagement surface engaging the finger of the actuator when the rack is in the advanced position to move the actuator from the first position to the second position.

19. The surgical device of claim 18, wherein the rack includes a second engagement surface positioned at a distal end of the elongate slot, the second engagement surface engaging the finger of the actuator when the rack is in the retracted position to move the actuator from the second position to the first position.

20. A powered handle assembly comprising:
an outer housing defining a cavity;
an inner housing secured within the cavity, the inner housing defining a longitudinally extending path through the inner housing;
a rack positioned along the longitudinally extending path of the inner housing, the rack including a first engagement surface and movable between retracted and advanced positions;
a drive assembly supported by the inner housing, the drive assembly engaged with the rack and operable to move the rack between the retracted and advanced positions;
a first switch supported within the cavity and coupled to the drive assembly, the first switch manually movable from an open position to a closed position;
an automatic retraction switch supported within the cavity, the automatic retraction switch coupled to the drive assembly and movable between open and closed positions;
an automatic retraction switch control mechanism including an actuator supported on the inner housing adjacent the automatic retraction switch, the actuator movable from a first position to a second position in response to movement of the rack from the retracted position to the advanced position to move the automatic retraction switch from the open position to the closed position; and
a retraction button supported on the housing, the retraction button engaged with the actuator and movable to move the actuator from the first position to the second position to cause retraction of the rack prior to movement of the rack to the advanced position;
wherein the first switch and the automatic retraction switch are configured such that when the first switch is in the closed position and the automatic retraction switch is in the open position, the drive assembly is actuated to move the rack towards the advanced position, and when the first switch is in the closed position and the automatic retract switch is in the closed position, the drive assembly is actuated to move the rack towards the retracted position.
